(19) United States
(12) Patent Application Publication
(10) Pub. No.: US 2022/0144754 A1
(43) Pub. Date: May 12, 2022

(54) PROCESS FOR PREPARATION OF MIDODRINE AND INTERMEDIATES THEREOF

(71) Applicant: CADILA HEALTHCARE LIMITED, Ahmedabad (IN)

(72) Inventors: Kumar Kamlesh Singh, Ahmedabad (IN); Sanjay Jagdish Desai, Ahmedabad (IN); Tadikonda Pratap V., Ahmedabad (IN); Mahesh Laljibhai Rupapara, Ahmedabad (IN)

(21) Appl. No.: 17/523,125

(22) Filed: Nov. 10, 2021

(30) Foreign Application Priority Data
Nov. 11, 2020 (IN) 202021049222

(51) Int. Cl. C07C 231/02; C07C 213/02

(52) U.S. Cl. CPC C07C 231/02; C07B 2200/13; C07C 213/02

(57) ABSTRACT

The invention relates to process for the preparation of midodrine or pharmaceutically acceptable salts thereof. The invention also relates to process for the preparation of intermediates of midodrine.

### FIELD OF THE INVENTION

The invention relates to process for the preparation of midodrine or pharmaceutically acceptable salts thereof. The invention also relates to process for the preparation of intermediates of midodrine.

### BACKGROUND OF THE INVENTION

The following discussion of background is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Midodrine hydrochloride (Ia) is a vasopressor/antihypotensive agent. Midodrine is a prodrug which forms an active metabolite, desglymidodrine (III) after oral administration. Desglymidodrine having a chiral centre, also exists in it racemic, (R) and (S) forms.

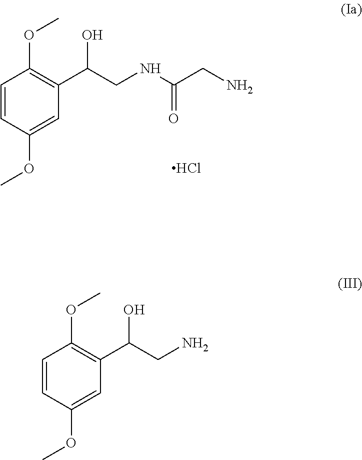 [SMILES: COc1ccc(OC)c(C(O)CNC(=O)CN)c1 ; Cl ; COc1ccc(OC)c(C(O)CN)c1]

U.S. Pat. Nos. 3,340,298 and 6,444,851 discloses midodrine and its intermediates and processes for preparation thereof.

International (PCT) Publication No. WO 1994/000593 and CN 101311162 discloses a process for preparation of intermediates of desglymidodrine.

International (PCT) Publication No. WO 2001/079154 discloses a process wherein the 2-chloro-1-(2,5-dimethoxyphenyl) ethane-1-one is reacted with sodium azide to obtain corresponding azido compound, followed by reduction to obtain desglymidodrine.

CN 102060719 and CN 105622434 discloses a process wherein 2,5-dimethoxy-benzaldehyde is reacted with nitromethane to obtain 2,5-dimethoxy-α-nitro methylbenzenemethanol, followed by reduction to obtain desglymidodrine.

The processes known in the art involves use of chemicals like hexamethylenetetramine, sodium azide and nitromethane that are hazardous and require cumbersome storage and handling conditions and procedures, which makes the known processes not much suitable for preparation of desglymidodrine, or thereby midodrine, commercially.

Therefore, there exists a need to provide a process for preparation of desglymidodrine, and from that midodrine or pharmaceutically acceptable salts thereof, which is efficient for commercial manufacturing.

Present invention provides a safe and cost-effective process for the preparation of desglymidodrine and midodrine or pharmaceutically acceptable salts thereof, which does not include any of the known hazardous chemicals and which is efficient for commercial manufacturing of desglymidodrine and midodrine or pharmaceutically acceptable salts thereof.

### SUMMARY OF THE INVENTION

In one general aspect, there is provided a process for the preparation of midodrine or pharmaceutically acceptable salts thereof, the process comprising:
(a) reacting 1,4-dimethoxybenzene (VII) with chloroacetyl chloride to obtain 2-chloro-1-(2,5-dimethoxyphenyl) ethan-1-one (VI);

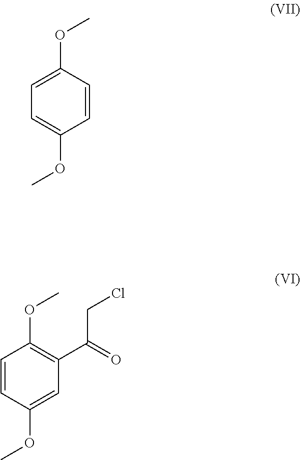

(b) reacting 2-chloro-1-(2,5-dimethoxyphenyl) ethan-1-one (VI) with phthalimide or a salt thereof to obtain 2-[2-(2,5-dimethoxyphenyl)-2-oxoethyl]-1H-isoindole-1,3(2H)-dione (V);

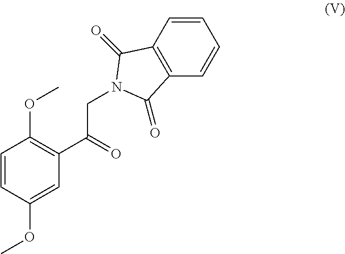 [SMILES: COc1ccc(OC)c(C(=O)CN2C(=O)c3ccccc3C2=O)c1]

(c) converting 2-[2-(2,5-dimethoxyphenyl)-2-oxoethyl]-1H-isoindole-1,3(2H)-dione (V) into 2-amino-1-(2′,5′-dimethoxyphenyl) ethanol (III);

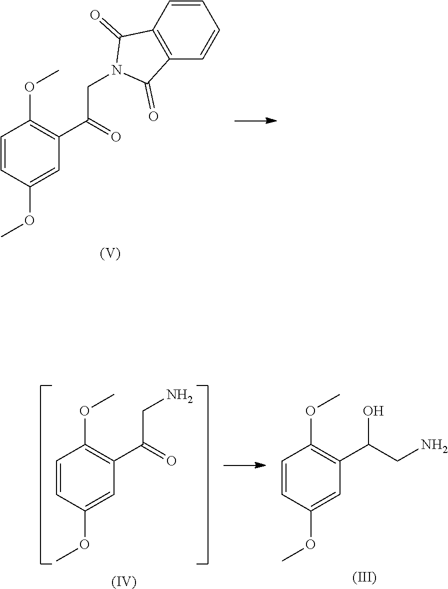

(d) converting 2-amino-1-(2′,5′-dimethoxyphenyl) ethanol (III) into midodrine; and

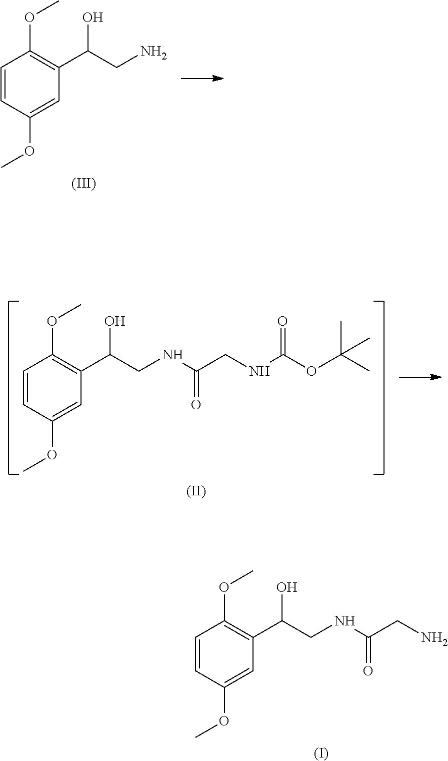

(e) optionally, converting midodrine into its pharmaceutically acceptable salt.

In another aspect, there is provided a process for the preparation of desglymidodrine, the process comprising:
(i) reacting 2-chloro-1-(2,5-dimethoxyphenyl) ethane-1-one (VI) with phthalimide or a salt thereof to obtain 2-(2-(2,5-dimethoxyphenyl)-2-oxoethyl) isoindoline-1,3-dione (V);

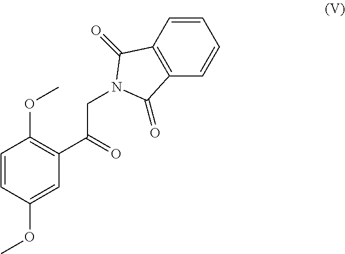 [SMILES: COc1ccc(OC)c(C(=O)CN2C(=O)c3ccccc3C2=O)c1]

(ii) reacting 2-(2-(2,5-dimethoxyphenyl)-2-oxoethyl) isoindoline-1,3-dione (V) with a base or an acid to obtain 2-amino-1-(2,5-dimethoxyphenyl) ethane-1-one (IV) or its salt; and

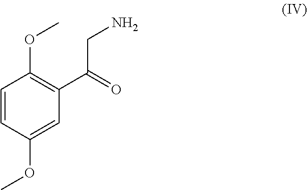

(iii) reducing 2-amino-1-(2,5-dimethoxyphenyl) ethane-1-one (IV) to obtain desglymidodrine (III)

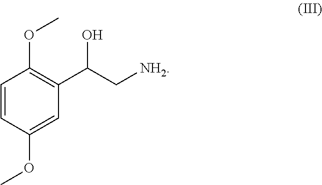

In another general aspect of the invention, there is provided a process for the preparation of desglymidodrine, the process comprising:
(i) reacting 1,4-dimethoxybenzene (VII) and chloroacetyl chloride in the presence of a Lewis acid to obtain 2-chloro-1-(2,5-dimethoxyphenyl) ethane-1-one (VI);

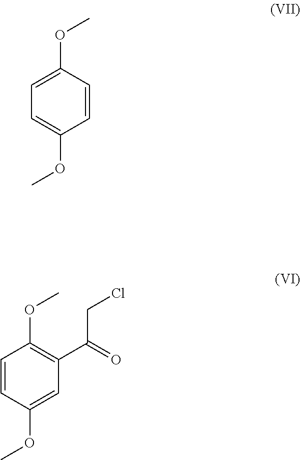

(ii) reacting 2-chloro-1-(2,5-dimethoxyphenyl) ethane-1-one (VI) with phthalimide or a salt thereof to obtain 2-(2-(2,5-dimethoxyphenyl)-2-oxoethyl) isoindoline-1,3-dione (V);

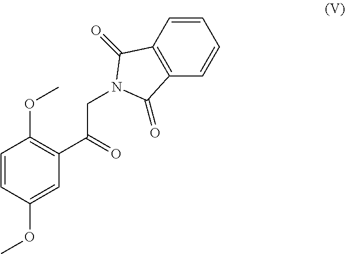 [SMILES: COc1ccc(OC)c(C(=O)CN2C(=O)c3ccccc3C2=O)c1]

(iii) reacting 2-(2-(2,5-dimethoxyphenyl)-2-oxoethyl) isoindoline-1,3-dione (V) with a base or an acid to obtain 2-amino-1-(2,5-dimethoxyphenyl) ethane-1-one (IV) or its salt; and

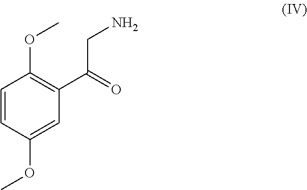

(iv) reducing the 2-amino-1-(2,5-dimethoxyphenyl) ethane-1-one (IV) or its salt to obtain desglymidodrine.

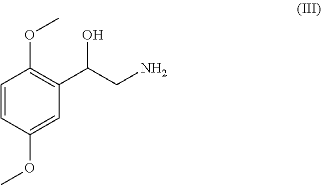

In another general aspect, there is provided a crystalline Form A of midodrine hydrochloride.

In another general aspect, there is provided a crystalline Form A of midodrine hydrochloride, characterized by X-ray powder diffraction pattern having at least two peaks expressed in degrees 2θ selected from 5.4°, 12.0°, 15.2°, and 21.6°±0.2°.

In another general aspect, there is provided a process for the preparation of crystalline Form A of midodrine hydrochloride, the process comprising:
(a) providing a solution of midodrine hydrochloride in one or more solvent;
(b) optionally treating with charcoal;
(c) cooling the solution; and
(d) removing the solvent from the solution to obtain crystalline Form-A of midodrine hydrochloride.

### BRIEF DESCRIPTION OF DRAWING

FIG. 1: X-ray powder diffraction pattern (XRD) of Midodrine Hydrochloride Form A.

FIG. 2: Differential Scanning calorimetry (DSC) of Midodrine Hydrochloride Form A.

FIG. 3: Thermogravimetric Analysis (TGA) of Midodrine Hydrochloride Form A.

### DETAILED DESCRIPTION OF THE INVENTION

The invention can be further understood in light of the description of the embodiments provided herein after. It is to be understood that the description, in no way, is intended to limit the scope of the invention to the expressly specified embodiments only. The equivalents and variants thereof or trivial modifications thereof which are apparently obvious to those skilled in the art, are also intended to be included within the scope of the present invention.

Detailed description of routine and conventional unit operations, which are easily understood by the skilled artisan, are not included herein. Such routine unit operations are to be construed as ordinarily understood and as routinely practiced by the person skilled in the field of the invention, unless otherwise specifically described.

In general, the terms ‘reacting’, ‘treating’ and ‘condensing’ are generally interchangeable and are used in their ordinary meaning as they are used in the field of the invention, unless defined specifically otherwise.

The terms ‘isolating’, ‘obtaining’ and ‘purifying’ are generally interchangeable, and include but not specifically limited to decantation, extraction, filtration, distillation, evaporation, lyophilisation, spray drying, crystallization, recrystallization or chromatographic operations.

The terms ‘pharmaceutically acceptable salts’, includes but not limited to hydrochloride, hydrobromide, hydroiodide, sulphate, phosphate, acetate, maleate, fumarate, tartrate, mandelate, besylate, tosylate salts. Of these salts, the hydrochloride is disclosed as being particularly preffered.

Removal of the solvent/undissolved solid and/or foreign particles can be done by distillation, distillation under reduced pressure, filtration, decantation, evaporation and centrifugation.

In one general aspect, there is provided a process for the preparation of midodrine or pharmaceutically acceptable salts thereof, the process comprising:
(a) reacting 1,4-dimethoxybenzene (VII) with chloroacetyl chloride to obtain 2-chloro-1-(2,5-dimethoxyphenyl) ethan-1-one (VI);

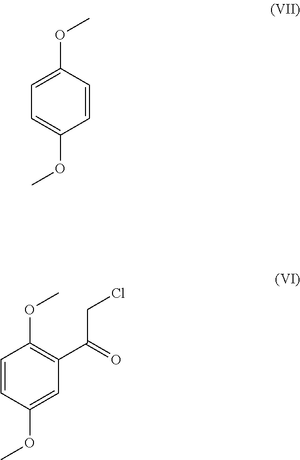

(b) reacting 2-chloro-1-(2,5-dimethoxyphenyl) ethan-1-one (VI) with phthalimide or a salt thereof salt to obtain 2-[2-(2,5-dimethoxyphenyl)-2-oxoethyl]-1H-isoindole-1,3(2H)-dione (V);

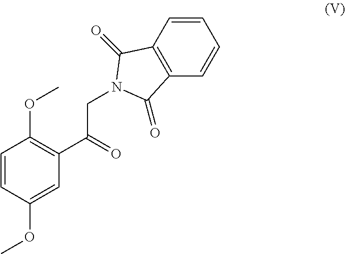 [SMILES: COc1ccc(OC)c(C(=O)CN2C(=O)c3ccccc3C2=O)c1]

(c) converting 2-[2-(2,5-dimethoxyphenyl)-2-oxoethyl]-1H-isoindole-1,3(2H)-dione (V) into 2-amino-1-(2′,5′-dimethoxyphenyl) ethanol (III);

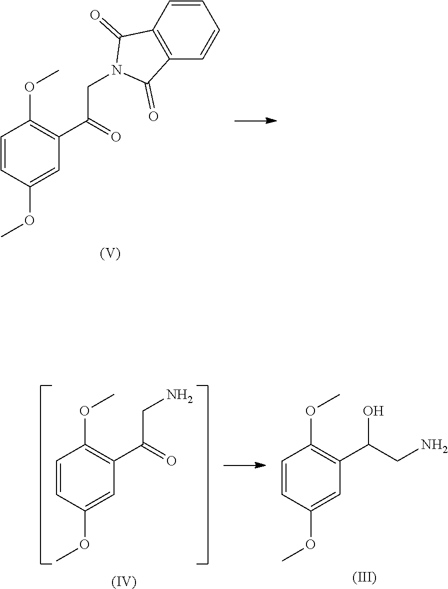

(d) converting 2-amino-1-(2′,5′-dimethoxyphenyl) ethanol (III) into midodrine; and

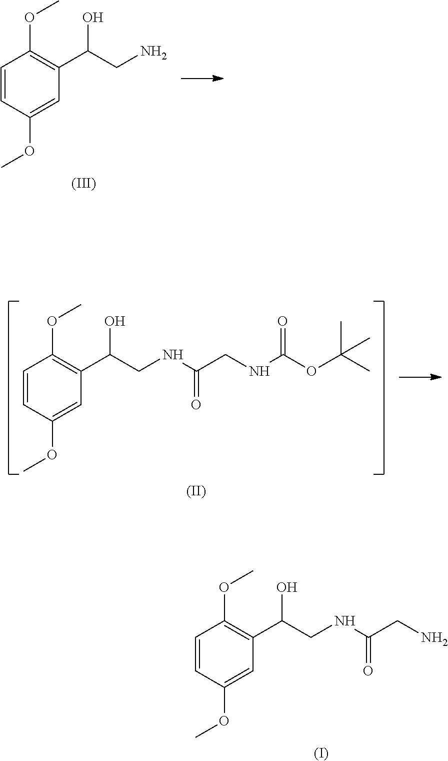

(e) optionally, converting midodrine into its pharmaceutically acceptable salt.

In general, 2-chloro-1-(2,5-dimethoxyphenyl) ethan-1-one, at step (a) can be prepared by reacting 1, 4-dimethoxy benzene with chloro acetyl chloride in presence of a Lewis acid selected from AlCl₃, AlBr₃, FeCl₃, and FeBr₃. Particularly, AlCl₃ can be used.

The reaction can be performed in presence of solvents selected from dichloroethane, dichloromethane, CCl₄, chloroform. Particularly, in presence of dichloromethane.

The reaction at step (a) can be performed at temperature range from 0° C. to 90° C. Particularly at 10° C. to 30° C.

At step (b), 2-[2-(2,5-dimethoxyphenyl)-2-oxoethyl]-1H-isoindole-1,3(2H)-dione can be prepared by reacting 2-chloro-1-(2,5-dimethoxyphenyl) ethan-1-one with phthalimide or a salt thereof.

Phthalimide or a salt thereof can be selected from potassium phthalimide or sodium phthalimide. Particularly, potassium phthalimide can be used.

The reaction can be performed in the presence of one or more solvent selected from toluene, benzene, xylene, N, N-dimethyl formamide, dimethyl sulfoxide. Particularly, the reaction can be performed in the presence of N, N-dimethyl formamide.

In general, the reaction at step (b) can be performed at temperature range from 20° C. to 130° C. Particularly, reaction can be performed at 60° C. to 70° C.

2-[2-(2,5-dimethoxyphenyl)-2-oxoethyl]-1H-isoindole-1,3(2H)-dione (V) can be converted into 2-amino-1-(2′,5′-dimethoxyphenyl) ethanol (III) at step (c), by first reacting 2-[2-(2,5-dimethoxyphenyl)-2-oxoethyl]-1H-isoindole-1,3(2H)-dione a base or an acid to obtain compound of Formula (IV) or a salt thereof and then reducing the compound of Formula (IV) by a reducing reagent to obtain 2-amino-1-(2′,5′-dimethoxyphenyl) ethanol (III).

The base can be selected from a group comprising sodium hydroxide, potassium hydroxide, lithium hydroxide, sodium carbonate, potassium carbonate, caesium carbonate, methylamine, ethylamine, dimethylamine, diethylamine, methylethylamine, triethylamine, ammonium hydroxide and hydrazine hydrate. Particularly, hydrazine hydrate or methyl amine can be used.

The acid can be selected from a group comprising, hydrochloric acid, hydrobromic acid, acetic acid, trichloroacetic acid and trifluoroacetic acid. Particularly, hydrochloric acid or acetic acid can be used.

In general, the solvents for the reaction can be selected from one or more of methanol, ethanol, propanol, isopropyl alcohol and butanol. Particularly, reaction can be performed using methanol as a solvent.

In general, the reaction can be performed at temperature range from 20° C. to 150° C. Particularly, reaction can be performed at 50° C. to 70° C.

The reaction can be conducted under an inert atmosphere e.g. under nitrogen atmosphere.

The compound of Formula (IV) or its salt can be isolated or directly insitu can be converted into 2-amino-1-(2′,5′-dimethoxyphenyl) ethanol (racemic-desglymidodrine). The compound of Formula (IV) or its salt can be hydrochloride, hydrobromide or hydroiodide. Particularly, salt can be hydrochloride.

The compound of Formula (IV) is reacted with a reducing agent in presence of one or more solvent to obtain 2-amino-1-(2′,5′-dimethoxyphenyl) ethanol (III).

Reducing agent for the reaction can be selected from lithium aluminium hydride, lithium borohydride, sodium borohydride, zinc borohydride, calcium borohydride, sodium cyanoborohydride, diisobutylaluminium hydride, L-selectride, diborane, diazene, aluminum hydride, Red-Al, and sodium triacetoxyborohydride. Particularly, sodium borohydride is used.

The reaction at step (c) can be performed in presence of base selected from alkali metal hydroxide for example sodium hydroxide, potassium hydroxide, lithium hydroxide. Particularly, sodium hydroxide is used.

The reaction can be performed in the presence of one or more solvent selected from C₁ to C₄-alcohol for example methanol, ethanol, propanol, isopropyl alcohol and butanol, water, N, N-dimethyl formamide, dimethylsulfoxide, dioxane, tetrahydrofuran or mixture thereof. Particularly, reaction can be performed using methanol as a solvent.

The reaction can be performed at temperature range −10° C. to 100° C. Particularly, the reaction can be performed at 0 to 10° C.

2-amino-1-(2′,5′-dimethoxyphenyl) ethanol (III), at step (d), is reacted with N-boc-glycine to obtain the compound of Formula (II).

The reaction can be performed in the presence of one or more solvent selected from dichloromethane, dichloroethane, chloroform, CCl₄, benzene, toluene, xylene. Particularly, dichloromethane can be used.

The amidation reaction is performed in the presence of coupling reagent selected from N, N′-dicyclohexylcarbodiimide (DCC), 1-ethyl-3-(3-dimethylaminopropyl) carbodiimide (EDC), and N, N′-diisopropyl carbodiimide (DIC). Particularly, N, N′-dicyclohexylcarbodiimide (DCC) can be used.

The reaction can be performed at temperature range −10° C. to 100° C. Particularly, the reaction can be performed at 0 to 50° C.

The compound of Formula (II) can either be isolated or can be converted directly into midodrine or its pharmaceutically acceptable salt.

The compound of Formula (II) either after isolation or insitu can be reacted with a pharmaceutically acceptable acid in presence of one or more solvent to obtain midodrine.

One or more solvent can be selected from water, isopropyl acetate, ethyl acetate, C₁-C₄-alcohol, for example methanol, ethanol, propanol, butanol, benzene and toluene.

Midodrine (I) can be reacted with pharmaceutically acceptable acid at step (e) to obtain pharmaceutically acceptable salt thereof.

Pharmaceutically acceptable acid for the steps (d) and (e) can be selected from hydrochloric acid, hydrobromic acid, hydroiodic acid, sulphuric acid, phosphoric acid, acetic acid, maleic acid, fumaric acid, tartaric acid, mandelic acid, benzene sulfonic acid, and toluene sulfonic acid. Preferably hydrochloric acid is used.

In another general aspect, there is provided a process for the preparation of desglymidodrine, the process comprising:
(i) reacting 2-chloro-1-(2,5-dimethoxyphenyl) ethane-1-one (VI) with phthalimide or a salt thereof to obtain 2-(2-(2,5-dimethoxyphenyl)-2-oxoethyl) isoindoline-1,3-dione (V);

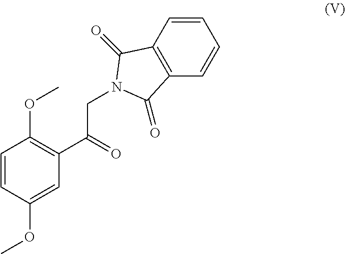 [SMILES: COc1ccc(OC)c(C(=O)CN2C(=O)c3ccccc3C2=O)c1]

(ii) reacting 2-(2-(2,5-dimethoxyphenyl)-2-oxoethyl) isoindoline-1,3-dione (V) with a base or an acid to obtain 2-amino-1-(2,5-dimethoxyphenyl) ethane-1-one (IV) or its salt; and

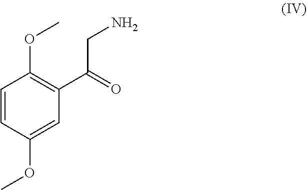

(iii) reducing 2-amino-1-(2,5-dimethoxyphenyl) ethane-1-one (IV) or its salt to obtain desglymidodrine (III)

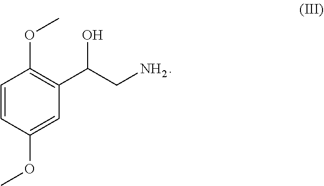

2-chloro-1-(2,5-dimethoxyphenyl) ethane-1-one can be prepared by reacting 1,4-dimethoxybenzene and chloroacetyl chloride in the presence of a Lewis acid.

Particularly, 1,4-dimethoxybenzene is reacted with 2-chloroacetyl chloride in the presence of a Lewis acid to obtain 2-chloro-1-(2,5-dimethoxyphenyl) ethane-1-one. Lewis acid for the reaction can be selected as mentioned herein above.

At step (i), phthalimide or a salt thereof can be selected from potassium phthalimide or sodium phthalimide. Particularly, potassium phthalimide can be used.

The reaction can be performed in the presence of one or more solvent selected from toluene, benzene, xylene, N, N-dimethyl formamide, dimethyl sulfoxide. Particularly, the reaction can be performed in the presence of N, N-dimethyl formamide.

In general, the reaction at step (i) can be performed at temperature range from 20° C. to 130° C. Particularly, reaction can be performed at 60° C. to 70° C.

At step (ii), 2-(2-(2,5-dimethoxyphenyl)-2-oxoethyl) isoindoline-1,3-dione (V) is reacted with a base or an acid to obtain 2-amino-1-(2,5-dimethoxyphenyl) ethane-1-one (IV) or its salt.

2-amino-1-(2,5-dimethoxyphenyl) ethane-1-one (IV) or its salt can be hydrochloride, hydrobromide or hydroiodide. Particularly, salt is hydrochloride salt.

The base can be selected from a group comprising sodium hydroxide, potassium hydroxide, lithium hydroxide, sodium carbonate, potassium carbonate, caesium carbonate, methylamine, ethylamine, dimethylamine, diethylamine, methylethylamine, triethylamine, ammonium hydroxide and hydrazine hydrate. Particularly, hydrazine hydrate or methyl amine can be used.

The acid can be selected from a group comprising, hydrochloric acid, hydrobromic acid, acetic acid, trichloroacetic acid and trifluoroacetic acid. Particularly, hydrochloric acid or acetic acid can be used.

In general, the solvents for the reaction can be selected from one or more of methanol, ethanol, propanol, isopropyl alcohol and butanol. Particularly, reaction can be performed using methanol as a solvent.

In general, the reaction can be performed at temperature range from 20° C. to 150° C. Particularly, reaction can be performed at 50° C. to 70° C.

At step (iii), the compound of Formula (IV) or it's salt can be reacted with a reducing agent in presence of one or more solvent to obtain 2-amino-1-(2′,5′-dimethoxyphenyl) ethanol (III).

Reducing agent for the reaction can be selected from lithium aluminium hydride, lithium borohydride, sodium borohydride, zinc borohydride, calcium borohydride, sodium cyanoborohydride, diisobutylaluminium hydride, L-selectride, diborane, diazene, aluminum hydride, Red-Al, and sodium triacetoxyborohydride. Particularly, sodium borohydride is used.

The reaction at step (c) can be performed in presence of base selected from alkali metal hydroxide for example sodium hydroxide, potassium hydroxide, and lithium hydroxide. Particularly, sodium hydroxide is used.

The reaction can be performed in the presence of one or more solvent as mentioned herein above.

The reaction can be performed at temperature range −10° C. to 100° C. Particularly, the reaction can be performed at 0 to 10° C.

In another general aspect of the invention, there is provided a process for the preparation of desglymidodrine, the process comprising:
(i) reacting 1,4-dimethoxybenzene (VII) and chloroacetyl chloride in the presence of a Lewis acid to obtain 2-chloro-1-(2,5-dimethoxyphenyl) ethane-1-one (VI);

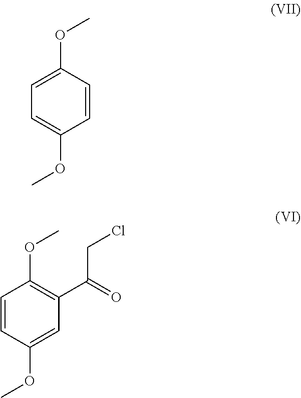

(ii) reacting 2-chloro-1-(2,5-dimethoxyphenyl) ethane-1-one (VI) with phthalimide or a salt thereof to obtain 2-(2-(2,5-dimethoxyphenyl)-2-oxoethyl) isoindoline-1,3-dione (V);

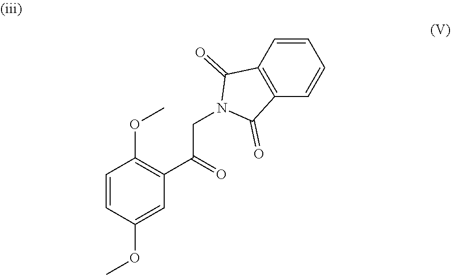 [SMILES: COc1ccc(OC)c(C(=O)CN2C(=O)c3ccccc3C2=O)c1]

(iv) reacting 2-(2-(2,5-dimethoxyphenyl)-2-oxoethyl) isoindoline-1,3-dione (V) with a base or an acid to obtain 2-amino-1-(2,5-dimethoxyphenyl) ethane-1-one (IV) or its salt; and

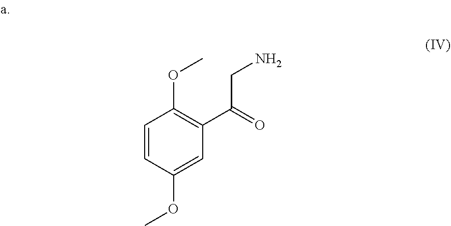

(v) reducing the 2-amino-1-(2,5-dimethoxyphenyl) ethane-1-one (IV) or its salt to obtain desglymidodrine.

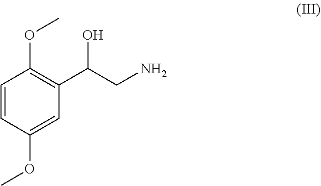

In another general aspects, corresponding bromo- and iodo-derivatives of 2-chloro-1-(2,5-dimethoxyphenyl)ethane-1-one can be prepared by reacting 1,4-dimethoxybenzene with haloacetyl bromide or haloacetyl iodide, respectively, and the 2-bromo-1-(2,5-dimethoxyphenyl)ethane-1-one or the 2-iodo-1-(2,5-dimethoxyphenyl)ethane-1-one obtained thereby can be reacted with phthalimide or a salt thereof to obtain 2-(2-(2,5-dimethoxyphenyl)-2-oxoethyl)isoindoline-1,3-dione.

Such variations of halogen as suggested herein are modifications of the embodiment(s) discussed with haloacetyl halide and 2-chloro-1-(2,5-dimethoxyphenyl) ethane-1-one and that is also an embodiment of the instant invention.

In general, 2-chloro-1-(2,5-dimethoxyphenyl) ethan-1-one, at step (i) can be prepared by reacting 1, 4-dimethoxy benzene with chloro acetyl chloride in presence of Lewis acid selected from AlCl₃, AlBr₃, FeCl₃, FeBr₃. Particularly, AlCl₃ can be used.

The reaction can be performed in presence of solvents selected from dichloroethane, dichloromethane, CCl₄, chloroform. Particularly, in presence of dichloromethane. The reaction at step (a) can be performed at temperature range from 0° C. to 90° C. Particularly at 10° C. to 30° C.

2-(2-(2,5-dimethoxyphenyl)-2-oxoethyl) isoindoline-1,3-dione can be obtained by reacting 2-chloro-1-(2,5-dimethoxyphenyl) ethane-1-one with phthalimide or a salt thereof, wherein the salts of phthalimide can be selected from a sodium salt or potassium salt.

The reaction can be performed in the presence of one or more solvent selected from toluene, benzene, xylene, N, N-dimethyl formamide, dimethyl sulfoxide. Particularly, the reaction can be performed in the presence of N, N-dimethyl formamide. The reaction can be performed at temperature range from 20° C. to 130° C. Particularly, reaction can be performed at 60° C. to 70° C.

In another general aspect, 2-amino-1-(2,5-dimethoxyphenyl) ethane-1-one is obtained at step (iii) by reacting 2-(2-(2,5-dimethoxyphenyl)-2-oxoethyl) isoindoline-1,3-dione with a base or an acid.

The base can be selected from a group comprising sodium hydroxide, potassium hydroxide, lithium hydroxide, sodium carbonate, potassium carbonate, caesium carbonate, methylamine, ethylamine, dimethylamine, diethylamine, methylethylamine, triethylamine, ammonium hydroxide and hydrazine hydrate. Particularly, hydrazine hydrate or methyl amine can be used.

In another general aspect, 2-amino-1-(2,5-dimethoxyphenyl) ethane-1-one or its salt is obtained by reacting 2-(2-(2,5-dimethoxyphenyl)-2-oxoethyl) isoindoline-1,3-dione with an acid.

2-amino-1-(2,5-dimethoxyphenyl) ethane-1-one or its salt can be hydrochloride, hydrobromide or hydroiodide. Particularly, salt is hydrochloride.

The acid can be selected from a group comprising, hydrochloric acid, hydrobromic acid, acetic acid, trichloroacetic acid and trifluoroacetic acid. Particularly, hydrochloric acid or acetic acid can be used.

The solvent for the reaction can be used particularly as mentioned herein above.

The reaction can be conducted under an inert atmosphere e.g., under nitrogen atmosphere.

Desglymidodrine can be obtained by reduction of 2-amino-1-(2,5-dimethoxyphenyl) ethane-1-one by a reducing agent comprising lithium aluminium hydride, lithium borohydride, sodium borohydride, zinc borohydride, calcium borohydride, sodium cyanoborohydride, diisobutylaluminium hydride, L-selectride, diborane, diazene, aluminum hydride and sodium borohydride. Particularly, sodium borohydride can be used.

In another general aspect of the invention, there is provided a process for the preparation of (R)- or (S)-enantiomers of desglymidodrine comprising:
(i) reacting 2-chloro-1-(2,5-dimethoxyphenyl) ethane-1-one with phthalimide or a salt thereof to obtain 2-(2-(2,5-dimethoxyphenyl)-2-oxoethyl) isoindoline-1,3-dione;
(ii) reacting 2-(2-(2,5-dimethoxyphenyl)-2-oxoethyl) isoindoline-1,3-dione with a base or an acid to obtain 2-amino-1-(2,5-dimethoxyphenyl) ethane-1-one; and
(iii) reducing the 2-amino-1-(2,5-dimethoxyphenyl) ethane-1-one in the presence of an enantioselective reducing agent to obtain (R)- or (S)-enantiomers desglymidodrine.

At step (i), 2-chloro-1-(2,5-dimethoxyphenyl) ethane-1-one can be reacted with potassium phthalimide or sodium phthalimide. Particularly, potassium phthalimide can be used.

At step (ii), base for the reaction, can be selected from sodium hydroxide, potassium hydroxide, lithium hydroxide, sodium carbonate, potassium carbonate, caesium carbonate, methylamine, ethylamine, dimethylamine, diethylamine, methylethylamine, triethylamine, ammonium hydroxide and hydrazine hydrate. Particularly, hydrazine hydrate or methyl amine can be used.

At step (ii), acid can be selected from a group comprising, hydrochloric acid, hydrobromic acid, acetic acid, trichloroacetic acid and trifluoroacetic acid. Particularly, hydrochloric acid or acetic acid can be used.

Specific (R)- or (S)-enantiomer of desglymidodrine can be obtained by using suitable chiral ligand or the enzyme.

2-amino-1-(2,5-dimethoxyphenyl) ethane-1-one can be reduced using an enantioselective or a chiral reducing agent, if desired, to obtain (R)- or (S)-enantiomers of desglymidodrine.

The enantioselective or the chiral reducing agent at step (iii) can be selected from a chiral catalyst or enantioselective reductive enzyme.

The chiral catalyst can be selected from a group comprising oxazaborolidine catalysts, e.g., Corey-Bakshi-Shibata (CBS) catalyst; transition metal catalyst with chiral ligand, e.g., ruthenium catalyst with BINAP ligands, Lithium aluminium hydride (LAH) modified with chiral alkoxide ligands or iridium complex of ferrocene-based phosphine-oxazoline ligand.

In another general aspect, there is provided a crystalline Form A of midodrine hydrochloride.

In another general aspect, there is provided a crystalline Form A of midodrine hydrochloride, characterized by X-ray powder diffraction pattern having at least two peaks expressed in degrees 2θ selected from 5.4°, 12.0°, 15.2°, and 21.6°±0.2°.

In another general aspect, there is provided a crystalline Form A of midodrine hydrochloride, which is characterized by X-ray powder diffraction pattern having characteristic peaks expressed in degrees 2θ selected from 5.4°, 10.7°, 12.0°, 13.4°, 15.2°, 16.1°, 17.2°, 20.7°, 21.6°, 22.1°, 23.8°, 24.2°, 25.4°, 26.5°, 27.0°, 27.6°, 29.4°, 30.6°, 31.7°, 34.3°, 34.7°, 36.4°, and 39.3°±0.2°.

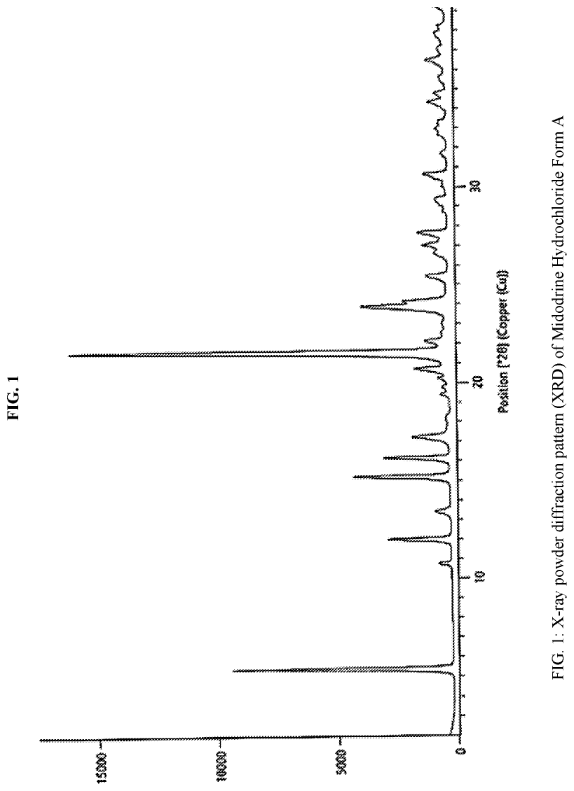

In another general aspect, there is provided a crystalline Form A of midodrine hydrochloride, characterized by X-ray powder diffraction pattern as depicted in FIG. 1.

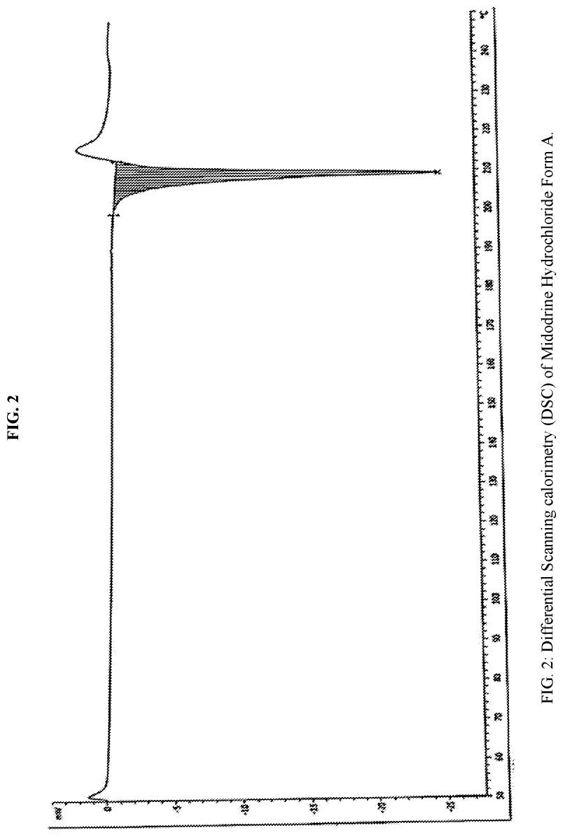

In general, the crystalline form A of Midodrine hydrochloride is characterized by Differential Scanning calorimetry (DSC) having endothermic peak at about 207.89° C. as shown in FIG. 2.

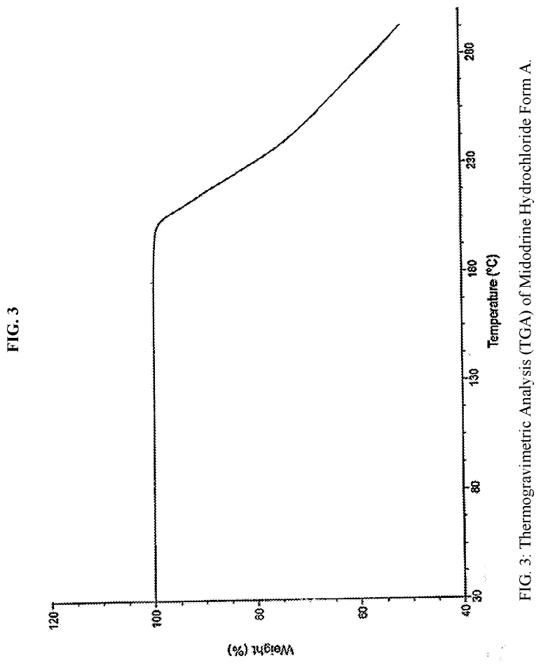

The crystalline Form A of midodrine hydrochloride is characterized by Thermogravimetric Analysis (TGA) curve as shown in FIG. 3.

In another general aspect, there is provided a process for the preparation of crystalline Form A of midodrine hydrochloride, the process comprising:
(a) providing a solution of midodrine hydrochloride in one or more solvents;
(b) optionally treating with charcoal;
(c) cooling the solution; and
(d) removing the solvent from the solution to obtain crystalline Form-A of midodrine hydrochloride.

In general, the one or more solvent at step (a) comprises one or more of water, ethyl acetate, isopropyl acetate, methanol, ethanol, propanol, isopropanol, and butanol.

In general, the step (a) of the reaction can be performed at temperature range selected from 0° C. to 100° C. Particularly, at temperature 20° C. to 60° C.

The solution at step (c) is then cooled to −5° C. to 20° C. Particularly, the solution is cooled to 5° C. to 15° C.

At step (d), removal of the solvent, can be done via distillation, distillation under reduced pressure, filtration, decantation, evaporation and centrifugation.

In another general aspect, there is provided a crystalline Form A of Midodrine hydrochloride having purity of about 99% or more, by area percentage of HPLC. In particular, the crystalline Form A having a purity of about 99.5% or more, more particularly, a purity of about 99.7% or more, by area percentage of HPLC.

The crystalline forms of Midodrine hydrochloride of the present invention may be characterized by Powder X-ray diffraction details as follow:

Instrumental Details

Characterization by X-Ray Powder Diffraction

The X-ray powder diffraction spectrum was measured under the following experimental conditions:

Instrument: X-Ray Diffractometer, Empyrean, Make: PANalytical.

| X-Ray: | Cu K alpha radiation | |
|---|---|---|
| Tension: | 45 | KV |
| Current: | 40 | mA |

| | |
|---|---|
| Divergence slit: | Automatic |

Incident Beam Side

| | |
|---|---|
| Off set: | 0.000 |
| Anti-scatter slit: | 1/2° |
| Receiving slit: | None |
| Detector: | PIXcellD-Medipix3 |
| Mode: | Scanning line detector (ID) |

Method Parameter

| | |
|---|---|
| Start position: | 2°2θ |
| End position: | 40°2θ |
| Step size: | 0.02° rad |
| Time per step: | 67.575 |
| Scan mode: | Continuous |

Differential Scanning calorimetry (DSC) analysis was performed using a Differential Scanning calorimeter using Model: DSC 3 Star E, Make: Mettler Toledo, Method: 50° C. to 250° C., Heating rate: 10° C. per minute, Nitrogen flow: 20 mL/min. About 1 to 3 mg of sample was placed at the pan and sealed with the sealing press. The instrument was programmed to heat at a heating rate of 10° C./min in the temperature range between 50° C. and 250° C.

Thermogravimetric Analysis (TGA) was performed using Thermogravimetric Analyser TGA Q500, make: TA analyzer instrument or equivalent. 3-10 mg of sample was placed in the sample pan. into the TG furnace. The instrument was programmed to heat at a heating rate of 10° C./min in the temperature range between 30° C. and 300° C., under nitrogen flow: For balance: 40 mL/min for Sample: 60 mL/min.

In another general aspect, there is provided a process for the preparation of Midodrine hydrochloride as shown in the following general reaction scheme (I):

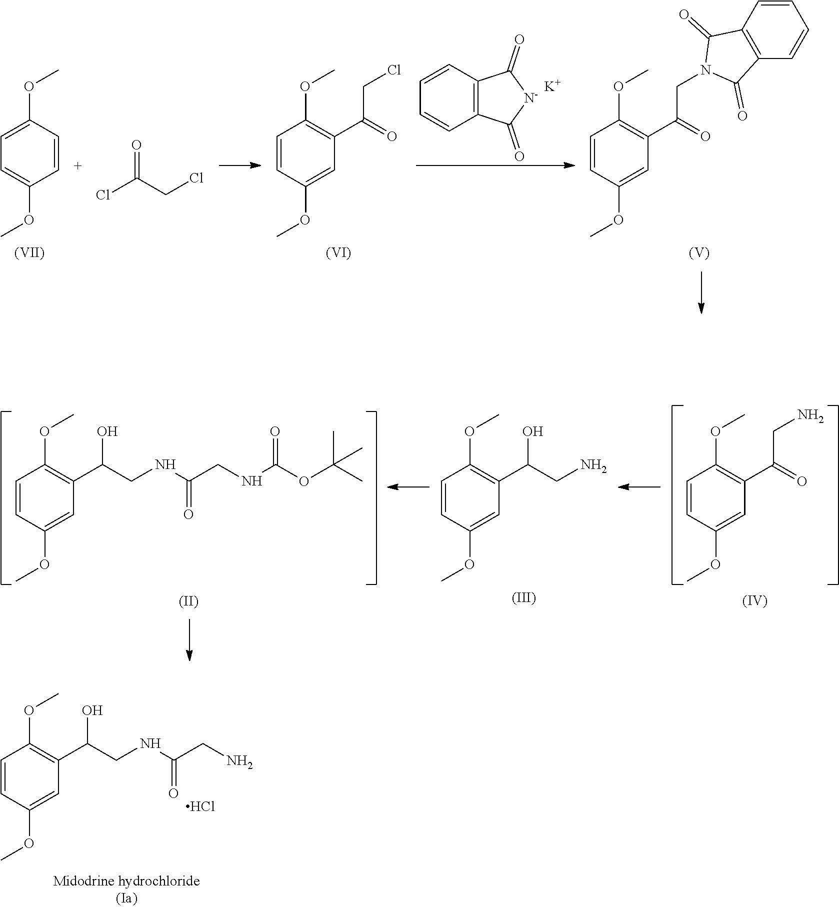

The embodiments of the invention can further be illustrated by following examples.

### EXAMPLES

### Example-1: 2-chloro-1-(2, 5-dimethoxyphenyl) ethane-1-one (VI)

In a round bottom flask, 106.16 g of 2-chloroacetyl chloride was added into a mixture of 125.3 g of anhydrous aluminum chloride in 800 mL dichloromethane under nitrogen atmosphere at 25-35° C. and then stirred at 40-45° C. for 1-2 hours. The reaction mixture was cooled to 0-10° C. and a solution of 100 g 1,4-dimethoxy benzene in 400 mL dichloromethane was added and stirred for 10-12 hours at 25-35° C. After completion of the reaction, aqueous HCl was added into the reaction mixture and stirred for 1-2 hours. The organic layer was separated and washed with water, aqueous NaOH and brine and then the solvent was distilled out at reduced pressure to obtain the titled compound (129 g).

### Example-2: 2-(2-(2, 5-dimethoxyphenyl)-2-oxoethyl) isoindoline-1, 3-dione (V)

In a round bottom flask, 100 g 2-chloro-1-(2,5-dimethoxyphenyl) ethane-1-one (VI) and 111 g potassium phthalimide were stirred in 1000 mL dimethylformamide at 95-100° C. for 1-2 hours. After completion of the reaction, the reaction mass was cooled to 10 to 20° C. and was poured into water (1000-1200 mL) and stirred for 1-2 hours. The solid was filtered, washed and dried to obtain the titled compound of Formula (V).

### Example-3: 2-amino-1-(2,5-dimethoxyphenyl) ethane-1-one (IV)

In a round bottom flask, Hydrazine hydrate (173 g, 80% in water) was added into a stirring mixture of 2-(2-(2,5-dimethoxyphenyl)-2-oxoethyl) isoindoline-1,3-dione (150 g) in methanol (2300 mL) at 20-25° C. and then stirred at 60-70° C. for an hour. The resultant mass was cooled to 25-35° C., 500 mL hydrochloric acid was added and stirred at 60-70° C. for 2-3 hours. After completion of the reaction, the resultant mass was cooled to 0-5° C. and filtered. The filtrate was distilled at reduced pressure to obtain 2-amino-1-(2,5-dimethoxyphenyl) ethane-1-one (IV).

### Example-4: Desglymidodrine (III)

After completion of reaction as per process described in Example-3, the resultant mass was cooled to 0-5° C. and filtered and 160 g sodium hydroxide and 17.4 g sodium borohydride were added, lot wise, under stirring at 0-10° C. The resultant mass was stirred for 30-60 min and after completion of the reaction, 150 mL hydrochloric acid was added and stirred for 15-30 min. The reaction mass was concentrated under reduced pressure to almost half of the initial volume and the remaining aqueous layer was washed with toluene and basified with aqueous sodium hydroxide under stirring. The resultant solid was fileted, washed and dried to obtain desglymidodrine (56 g).

### Example-5: Preparation of Midodrine hydrochloride (Ia)

### Stage-1: Preparation of 2-chloro-1-(2,5-dimethoxyphenyl)ethan-1-one (VI)

In a round bottom flask, 600 mL dichloromethane, 96.50 g anhydrous AlCl₃, were taken under nitrogen atmosphere and cooled to 0 to 10° C. 261.55 g chloro acetyl chloride was added and stirred for 15±5 minutes at 0 to 10° C. then the reaction mixture was stirred at 37 to 42° C. for 1-2 hours and then cooled to 0 to 10° C. A solution of 100 g 1, 4-dimethoxy benzene in 200 mL dichloromethane was added and stirred for 15±5 minutes at 0 to 10° C. The reaction mixture was stirred for 24 to 25 hour under nitrogen atmosphere at 17 to 23° C. After completion of the reaction, the reaction mixture was added into HCl in water and stirred at 0 to 20° C. for 15 to 20 minutes and then stirred at 25 to 35° C. for 20 to 25 minutes. The layers were separated and the organic layer was washed with sodium bicarbonate solution and then with water. The solvent was removed from the organic layer and 200 mL methanol was added into the reaction mixture at below 50° C. and stirred for 15 to 20 minutes and then it was removed under vacuum at below 50° C. Again 250 mL methanol was added at below 50° C. and the reaction mixture was stirred at 55 to 60° C. for 30 to 35 minutes. The reaction mixture was then cooled and stirred at 25 to 35° C. for an hour and then at 0 to 10° C. for an hour. The solid was filtered and washed with pre-cooled methanol and then dried under vacuum (Vacuum: NLT 700 mmHg) for 8 to 9 hours to obtain 2-chloro-1-(2,5-dimethoxyphenyl) ethan-1-one.

### Stage-2: Preparation of 2-[2-(2,5-Dimethoxyphenyl)-2-oxoethyl]-1H-isoindole-1,3(2H)-dione (V)

100 g of 2-chloro-1-(2,5-dimethoxyphenyl) ethan-1-one, 300 mL DMF and 94.92 g potassium phthalimide were taken in a round bottom flask and stirred at 25 to 35° C. under nitrogen atmosphere for 2-3 hours and then stirred at 60 to 70° C. for an hour. After completion of the reaction, the reaction mixture was cooled to 25 to 35° C. and further cooled to 0 to 20° C. and 400 mL water was added and stirred at 20 to 30° C. for an hour. The solid was filtered and washed with water. The wet cake and 450 mL methanol were taken in another flask and stirred at 50 to 60° C. for an hour and then cooled to 20 to 30° C. and stirred for an hour. The solid was filtered and washed with methanol and then dried under vacuum for 8 to 9 hours at 50 to 60° C. to obtain 2-[2-(2, 5-dimethoxyphenyl)-2-oxoethyl]-1H-isoindole-1,3(2H)-dione.

### Stage-3: Preparation of 2-Amino-1-(2′,5′-dimethoxyphenyl)ethanol (III)

In a round bottom flask, 800 mL methanol, 100 g 2-[2-(2,5-Dimethoxyphenyl)-2-oxoethyl]-1H-isoindole-1,3(2H)-dione, 76.94 g of hydrazine hydrate (80%) and 200 mL methanol were added and stirred for 10 to 15 minutes at 25 to 35° C. The reaction mixture was stirred at 60 to 65° C. for 2 to 3 hours. After completion of the reaction, the reaction mixture was cooled to 25 to 40° C. and 170 g concentrated hydrochloric acid was added at 25 to 40° C. The reaction mixture was stirred at 50 to 60° C. for 2 to 3 hours and then stirred at 0 to 5° C. for an hour. The solid was filtered and washed with pre-cooled methanol. The filtrate was taken in another flask and methanol was distilled out under vacuum at below 60° C. then 500 mL isopropyl alcohol was added and stirred at 55 to 65° C. for 30 to 35 minutes and then stirred for 1 to 2 hours at 0 to 10° C. The solid was filtered and washed with isopropyl alcohol and dried to obtain a wet cake of compound of Formula (IV). The wet cake and 250 mL methanol were taken in another flask and stirred for 10 to 15 minutes at 25 to 35° C. The reaction mixture was cooled to 0 to 10° C. and a solution of 18.44 g sodium hydroxide and 13.37 g sodium borohydride in 85 mL water was added at 0 to 10° C. and stirred for an hour at 0 to 10° C. 90 g concentrated HCl solution in 77 mL water was added at 0 to 30° C. and stirred for 15±5 minutes and methanol was distilled under vacuum below 50° C. 170 mL water and 170 mL toluene were added and stirred for 30-35 minutes at 25 to 35° C. The reaction mixture was settled and layers were separated. The aqueous layer was taken in another flask and a solution of 57 g NaOH in 85 mL water was added into the reaction mixture at 0 to 5° C. and stirred for 2 hours then the solid was filtered and dried under vacuum for 2-3 hours at 25 to 35° C. 42 g of this crude solid and 462 mL isopropyl acetate were taken in another flask and stirred for 30 to 35 minutes at 60 to 70° C. The reaction mixture was filtered and washed with isopropyl acetate at 60 to 70° C. The filtrate was taken in another flask and stirred at 50 to 60° C. for 10 to 15 minutes then cooled to 25 to 35° C. and stirred at this temperature for 30 to 35 minutes and after that stirred at 0 to 5° C. for 2 to 3 hours. The solid was filtered and washed with pre-cooled isopropyl acetate and then dried under vacuum for 2-3 hours at 25 to 35° C. and further the solid was dried in vacuum tray dryer for 8 to 9 hours at 50 to 60° C. temperature to obtain 2-amino-1-(2′, 5′-dimethoxyphenyl) ethan-1-ol.

### Stage-4: Preparation of midodrine hydrochloride (Ia)

10 g of 2-amino-1-(2′, 5′-dimethoxyphenyl) ethan-1-ol, 9.76 g of tert-butoxy carbonyl amino acetic acid (N-boc-glycine) and 75 ml dichloro methane were taken in a round bottom flask and the reaction mixture was stirred for 10 minutes at 25-35° C. than cooled to 0-10° C. 11.5 g N, N′-dicyclohexylcarbodiimide solution was dissolved in 25 ml dichloromethane at 0-10° C. and stirred for 2 hours at 20-30° C. The reaction mixture was stirred for 2 hours at 40-45° C. and then cooled and stir for 75 minutes at 5-15° C. and then filtered. The filtrate was washed with 20 mL of 10% sodium carbonate solution and then with 20 mL water and then stirred for 15 minutes. The layers were separated and solvent was removed under vacuum to remove traces to give 18 g residue. 10 ml isopropyl acetate was added and distilled out under vacuum. Then 75 mL isopropyl acetate was added and cooled to 20-25° C. and a solution of 71 mL isopropyl acetate hydrochloride and 8 mL water at 20-25° C. The reaction mixture was filtered to obtain a crude wet cake. In another flask, 24 g wet cake, 260 mL isopropyl alcohol and 45.5 mL water were taken and stirred for 15 minutes at 40-50° C. then 0.65 g activated charcoal was added and stirred for 15 minutes at 40-50° C. The reaction mixture was filtered through celite and the filtrate was cooled and stirred at 25-35° C. for an hour and further cooled and stirred at 5-15° C. for an hour and filtered to obtain a solid. The solid was dried at 25-35° C. for 2 hours then at 55-60° C. for 2 hour and at 85-90° C. for 14 hours to obtain Midodrine hydrochloride.

### Example-6: Synthesis of Form A of Midodrine Hydrochloride

In a round bottom flask, 20 g Midodrine Hydrochloride, 200 mL isopropyl alcohol and 40 mL water were taken and stirred for 15 to 30 minutes at 40-50° C. then 0.70 g activated charcoal was added and stirred for 15 to 30 minutes at 40-50° C. The reaction mixture was filtered through celite and the filtrate was stirred at 25-35° C. for an hour and further cooled and stirred to 5-15° C. for an hour and filtered to obtain a solid. The solid was dried at 25-35° C. for 2 hours then at 55-60° C. for 2 to 3 hour and at 85-90° C. for 14 to 15 hours to obtain Midodrine hydrochloride Form A.

XRD 2θ: 5.4°, 10.7°, 12.0°, 13.4°, 15.2°, 16.1°, 17.2°, 20.7°, 21.6°, 22.1°, 23.8°, 24.2°, 25.4°, 26.5°, 27.0°, 27.6°, 29.4°, 30.6°, 31.7°, 34.3°, 34.7°, 36.4°, and 39.3°±0.2°.

DCS: Endothermic peak at about 207.89° C.

While the present invention has been described in terms of a few specific embodiments, modification and equivalents thereof, in light of the teaching and disclosure of the present invention, that are apparent to the skilled artisan, are to be construed as included within the scope of the invention.

We claim:

1. A process for the preparation of midodrine or pharmaceutically acceptable salts thereof, the process comprising:
(a) reacting 1,4-dimethoxybenzene (VII) with chloroacetyl chloride to obtain 2-chloro-1-(2,5-dimethoxyphenyl)ethan-1-one (VI); 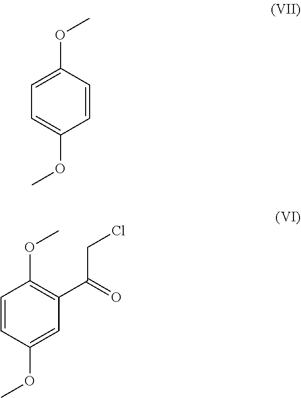
(b) reacting 2-chloro-1-(2,5-dimethoxyphenyl)ethan-1-one (VI) with phthalimide or a salt thereof to obtain 2-[2-(2,5-dimethoxyphenyl)-2-oxoethyl]-1H-isoindole-1,3(2H)-dione (V); 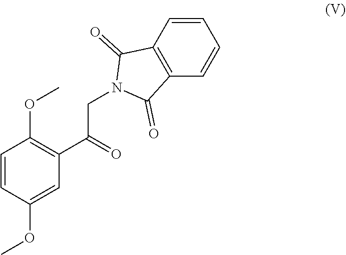 [SMILES: COc1ccc(OC)c(C(=O)CN2C(=O)c3ccccc3C2=O)c1]
(c) converting 2-[2-(2,5-dimethoxyphenyl)-2-oxoethyl]-1H-isoindole-1,3(2H)-dione (V) into 2-amino-1-(2′,5′-dimethoxyphenyl)ethanol (III); 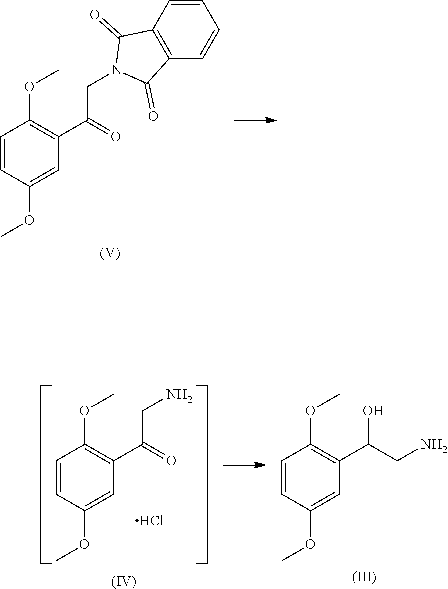
(d) converting 2-amino-1-(2′,5′-dimethoxyphenyl)ethanol (III) into midodrine; and 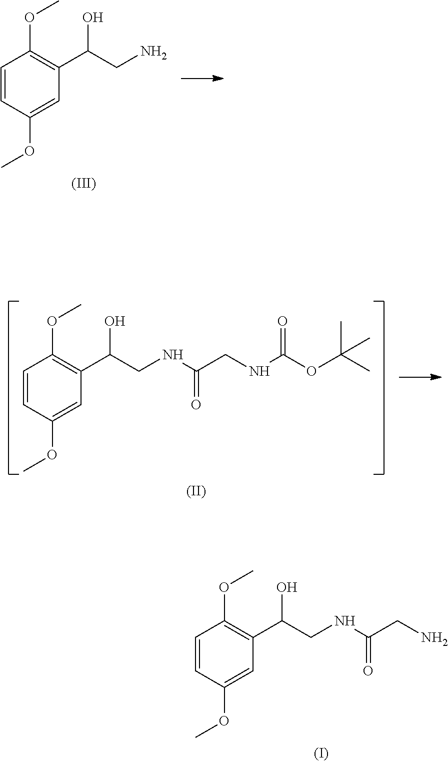
(e) optionally, converting midodrine into its pharmaceutically acceptable salt.

2. The process according to claim 1, wherein the reaction at step (a) is performed in the presence of a Lewis acid selected from AlCl₃, AlBr₃, FeCl₃, and FeBr₃.

3. The process according to claim 1, wherein the reaction at step (b) is performed using potassium phthalimide or sodium phthalimide.

4. The process according to claim 1, wherein the conversion of the compound of Formula (V) into the compound of Formula (IV) at step (c) is performed in the presence of a base or an acid.

5. The process according to claim 4, wherein the base is selected from sodium hydroxide, potassium hydroxide, lithium hydroxide, sodium carbonate, potassium carbonate, caesium carbonate, methylamine, ethylamine, dimethylamine, diethylamine, methylethylamine, triethylamine, ammonium hydroxide, and hydrazine hydrate.

6. The process according to claim 4, wherein the acid is selected from hydrochloric acid, hydrobromic acid, acetic acid, trichloroacetic acid, and trifluoroacetic acid.

7. The process according to claim 1, wherein the conversion of the compound of Formula (IV) into the compound of Formula (III) at step (c) is performed in the presence of a reducing agent selected from lithium aluminium hydride, lithium borohydride, sodium borohydride, zinc borohydride, calcium borohydride, sodium cyanoborohydride, diisobutylaluminium hydride, L-selectride, diborane, diazene, aluminum hydride, Red-Al, and sodium triacetoxyborohydride.

8. The process according to claim 1, wherein the conversion of the compound of Formula (III) into the compound of Formula (II) at step (d) is performed by reacting 2-amino-1-(2′,5′-dimethoxyphenyl)ethanol (III) with N-boc-glycine.

9. The process according to claim 8, wherein the conversion is performed in the presence of a coupling reagent selected from N,N′-dicyclohexylcarbodiimide (DCC), 1-ethyl-3-(3-dimethylaminopropyl)carbodiimide (EDC), and N,N′-diisopropyl carbodiimide (DIC).

10. The process according to claim 8, wherein the conversion is performed in the presence of one or more solvents selected from dichloromethane, dichloroethane, chloroform, CCl₄, benzene, toluene, and xylene.

11. The process according to claim 1, wherein the conversion of the compound of Formula (II) into the compound of Formula (I) at step (d) is performed by reacting the compound of Formula (II) with an acid selected from hydrochloric acid, hydrobromic acid, hydroiodic acid, sulphuric acid, phosphoric acid, acetic acid, maleic acid, fumaric acid, tartaric acid, mandelic acid, benzene sulfonic acid, and toluene sulfonic acid.

12. A process for the preparation of desglymidodrine of Formula (III), 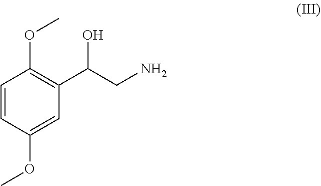
the process comprising:
(i) reacting 2-chloro-1-(2,5-dimethoxyphenyl)ethane-1-one (VI) with phthalimide or a salt thereof to obtain 2-(2-(2,5-dimethoxyphenyl)-2-oxoethyl)isoindoline-1,3-dione (V); 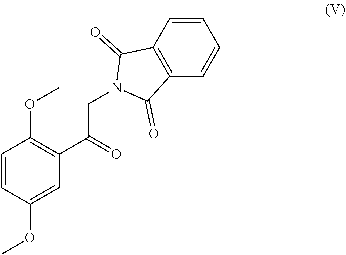 [SMILES: COc1ccc(OC)c(C(=O)CN2C(=O)c3ccccc3C2=O)c1]
(ii) reacting 2-(2-(2,5-dimethoxyphenyl)-2-oxoethyl)isoindoline-1,3-dione (V) with a base or an acid to obtain 2-amino-1-(2,5-dimethoxyphenyl)ethane-1-one (IV) or its salt; and 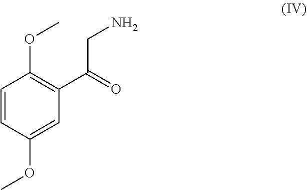
(iii) reducing 2-amino-1-(2,5-dimethoxyphenyl)ethane-1-one (IV) or its salt to obtain desglymidodrine (III).

13. The process according to claim 12, wherein 2-chloro-1-(2,5-dimethoxyphenyl)ethane-1-one (VI) at step (i) is reacted with potassium phthalimide or sodium phthalimide.

14. The process according to claim 12, wherein the base at step (ii) is selected from sodium hydroxide, potassium hydroxide, lithium hydroxide, sodium carbonate, potassium carbonate, caesium carbonate, methylamine, ethylamine, dimethylamine, diethylamine, methylethylamine, triethylamine, ammonium hydroxide, and hydrazine hydrate.

15. The process according to claim 12, wherein the acid at step (ii) is selected from hydrochloric acid and acetic acid.

16. The process according to claim 12, wherein the reduction reaction at step (iii) is performed using a reducing agent selected from lithium aluminium hydride, lithium borohydride, sodium borohydride, zinc borohydride, calcium borohydride, sodium cyanoborohydride, diisobutylaluminium hydride, L-selectride, diborane, diazene, aluminum hydride, Red-Al, and sodium triacetoxyborohydride.

17. A process for the preparation of desglymidodrine of Formula (III), 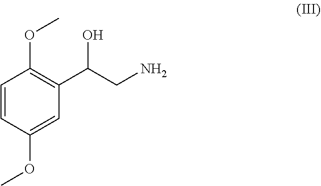
the process comprising:
(i) reacting 1,4-dimethoxybenzene (VII) and chloroacetyl chloride in the presence of a Lewis acid to obtain 2-chloro-1-(2,5-dimethoxyphenyl) ethane-1-one (VI); 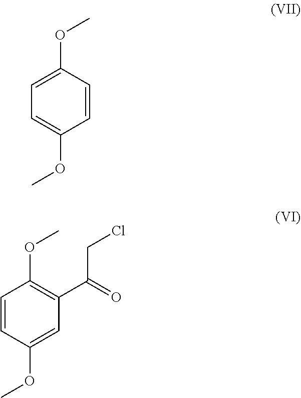
(ii) reacting 2-chloro-1-(2,5-dimethoxyphenyl) ethane-1-one (VI) with phthalimide or a salt thereof to obtain 2-(2-(2,5-dimethoxyphenyl)-2-oxoethyl) isoindoline-1,3-dione (V); 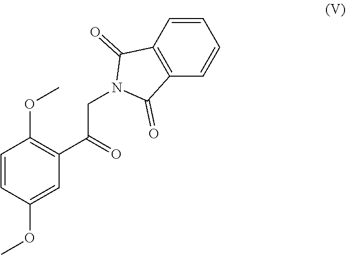 [SMILES: COc1ccc(OC)c(C(=O)CN2C(=O)c3ccccc3C2=O)c1]
(iii) reacting 2-(2-(2,5-dimethoxyphenyl)-2-oxoethyl) isoindoline-1,3-dione (V) with a base or an acid to obtain 2-amino-1-(2,5-dimethoxyphenyl) ethane-1-one (IV) or its salt; and 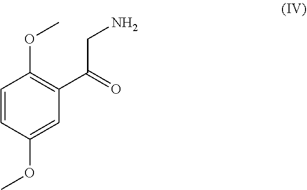
(iv) reducing the 2-amino-1-(2,5-dimethoxyphenyl) ethane-1-one (IV) or its salt to obtain desglymidodrine.

18. The process according to claim 17, wherein the reaction at stage (i) is performed in the presence of a Lewis acid selected from AlCl₃, AlBr₃, FeCl₃, and FeBr₃.

19. The process according to claim 17, wherein the reaction at stage (ii) is performed using potassium phthalimide or sodium phthalimide.

20. The process according to claim 17, wherein the base at step (iii) is selected from sodium hydroxide, potassium hydroxide, lithium hydroxide, sodium carbonate, potassium carbonate, caesium carbonate, methylamine, ethylamine, dimethylamine, diethylamine, methylethylamine, triethylamine, ammonium hydroxide, and hydrazine hydrate.

21. The process according to claim 17, wherein the acid at step (iii) is selected from hydrochloric acid, hydrobromic acid, acetic acid, trichloroacetic acid, and trifluoroacetic acid.

22. The process according to claim 17, wherein the reduction of 2-amino-1-(2,5-dimethoxyphenyl) ethane-1-one (IV) or its salt at stage (iv) is performed in the presence of a reducing agent selected from lithium aluminium hydride, lithium borohydride, sodium borohydride, zinc borohydride, calcium borohydride, sodium cyanoborohydride, diisobutylaluminium hydride, L-selectride, diborane, diazene, aluminum hydride, Red-Al, and sodium triacetoxyborohydride.

23. A crystalline Form A of midodrine hydrochloride, characterized by X-ray powder diffraction pattern having at least two peaks expressed in degrees 2θ selected from 5.4°, 12.0°, 15.2°, and 21.6°±0.2°.

24. A process for the preparation of crystalline Form A of midodrine hydrochloride, the process comprising:
(a) providing a solution of midodrine hydrochloride in one or more solvents;
(b) optionally treating with charcoal;
(c) cooling the solution; and
(d) removing the solvent from the solution to obtain crystalline Form-A of midodrine hydrochloride.

25. The process according to claim 24, wherein the one or more solvent at step (a) is selected from water, ethyl acetate, isopropyl acetate, methanol, ethanol, propanol, isopropanol, and butanol.

26. The process according to claim 24, wherein the cooling at step (c) is performed at −5° C. to 20° C.

27. The process according to claim 24, wherein the removal of the solvent at step (d) is performed by one or more of distillation, distillation under reduced pressure, filtration, decantation, evaporation and centrifugation.
